(19) United States
(12) Patent Application Publication
(10) Pub. No.: US 2005/0155091 A1
(43) Pub. Date: Jul. 14, 2005

(54) Prkag3 gene promoter and uses thereof

(76) Inventor: Thomas Svensson, Floda (SE)

Correspondence Address: FISH & RICHARDSON P.C., PO BOX 1022, MINNEAPOLIS, MN, 55440-1022, US

(21) Appl. No.: 10/503,039

(22) PCT Filed: Jan. 31, 2003
(86) PCT No.: PCT/IB03/00762

Related U.S. Application Data
(60) Provisional application No. 60/353,429, filed on Feb. 1, 2002.

(51) Int. Cl. A01K 67/00; C12Q 1/68

(52) U.S. Cl. 800/13; 536/24.1; 435/320.1; 435/455; 435/6; 536/23.5

(57) ABSTRACT

The invention provides an isolated human Prkag3 promoter. Expression constructs containing the Prkag3 promoter also are provided, as are methods of using such expression constructs to direct expression of a heterologous coding sequence. Host cells containing an expression construct of the invention are provided, as well as methods of using such cells to screen for compounds that transcriptionally modulate the activity of a Prkag3 promoter.

### TECHNICAL FIELD

This invention relates to promoters useful to drive expression of genes of interest in a tissue-specific manner, and more particularly, to skeletal muscle specific promoters, expression cassettes containing such promoters and their use as drug screening tools, and cells and organisms containing such expression cassettes.

### BACKGROUND

AMP-activated kinase (AMPK) has a key role in regulating energy metabolism in eukaryotic cells and is homologous to the SNF1 kinase in yeast (Hardie D. G. et al., 1998, Annu. Rev. Biochem., 67: 821; Kemp B E. et al., 1999, Trends. Biochem. Sci., 24(1): 22-5). AMPK is composed of three subunits: a catalytic α-chain and two regulatory subunits, β and γ. AMPK is activated by an increase in the ratio of AMP to ATP (AMP:ATP). Activated AMPK turns on ATP-producing pathways and inhibits ATP-consuming pathways. AMPK also can inactivate glycogen synthase, the key regulatory enzyme of glycogen synthesis, by phosphorylation (Hardie et al. 1998 supra). Several isoforms of the three different AMPK subunits are present in mammals. In humans, Prkaa1 and Prkaa2 encode the α1 and α2 subunits, Prkab1 and Prkab2 encode the β1 and β2 subunits, and Prkag1, Prkag2 and Prkag3 encode the γ1, γ2 and γ3 subunits, respectively.

Milan D. et al. (2001, Science, 288: 1248-51) identified a nonconservative substitution of an arginine to a glutamine at position 200 (R200Q) in the Hampshire pig Prkag3 gene, which is responsible for the dominant RN⁻ phenotype that causes high glycogen content in skeletal muscle. Loss-of-function mutations in the homologous gene in yeast (SNF4) cause defects in glucose metabolism, including glycogen storage. Milan et al. further found that the expression of the Prkag3 gene is muscle-specific and that the AMPK activity in muscle extracts was about three times higher in normal rn⁺ pigs than in RN⁻ pigs, both in the presence and absence of AMP. The distinct phenotype of the RN⁻ mutation indicates that Prkag3 plays a key role in the regulation of energy metabolism in skeletal muscle.

AMPK is recognized as a major regulator of lipid biosynthetic pathways due to its role in the phosphorylation and inactivation of key enzymes such as acetyl-CoA carboxylase (ACC) (Hardie & Carling, 1997, Eur. J. Biochem., 246: 259-273). More recent data strongly suggest that AMPK has a wider role in metabolic regulation (Winder & Hardie, 1999, Am. J. Physiol., 277: E1-E10), including fatty acid oxidation, muscle glucose uptake (Hayashi T. et al., 1998, Diabetes, 47: 1369-1373; Merrill G F. et al., 1997, Am. J. Physiol., 273: E1107-E1112; Goodyear L. J., 2000, Exerc. Sport Sci. Rev., 28: 113-116), expression of cAMP-stimulated gluconeogenic genes such as PEPCK and G6 Pase (Lochhead P. A. et al., 2000, Diabetes, 49: 896-903), and glucose-stimulated genes associated with hepatic lipogenesis, including fatty acid synthase (FAS), Spot-14 (S14), and L-type pyruvate kinase (Foretz M. et al., 1998, J. Biol. Chem., 273: 14767-14771). Chronic activation of AMPK also may induce the expression of muscle hexokinase and glucose transporters (Glut4), mimicking the effects of extensive exercise training (Holmes B. F. et al., 1999, J. Appl. Physiol., 87: 1990-1995). Thus, it has been predicted that AMPK activation would be a good approach to treat type 2 diabetes (Wmder & Hardie 1999, supra).

Zhou G et al. (2001, J. Clin. Invest., 108: 1167-1174) provided evidence that the elusive target of metformin's (a widely used drug for treatment of type 2 diabetes) actions is activated AMPK. In studies performed in isolated hepatocytes and rat skeletal muscles, it was demonstrated that metformin leads to AMPK activation, accompanied by an inhibition of lipogenesis (due to inactivation of acetyl-CoA carboxylase and suppression of lipogenic enzyme expression), suppression of the expression of SREBP-1 (a central lipogenic transcription factor), and a modest stimulation of skeletal muscle glucose uptake. Similar hepatic effects are seen in metformin-treated rats. Based on the use of a newly discovered AMPK inhibitor, their data suggest that the ability of metformin to suppress glucose production in hepatocytes requires AMPK activation.

In skeletal muscle, AMPK is part of the signalling system in contraction- and hypoxia-regulated glucose uptake and a mediator of leptin stimulated fatty-acid oxidation (Mu J. et al., 2001, Cell, 7: 1085-1094, Minokoshi Y. et al., 2002, Nature, 415: 339-343).

The cDNA encoding the human γ3 subunit has been cloned and characterized (WO 01/20003, Milan et al. 2000, GenBankAF214519, Cheung P. C. et al., 2000, Biochem J, 346: 659-69, GenBank AF249977). Genetic variants of the human Prkag3 gene encoding the AMPK γ3 subunit also have been identified (WO 01/77305).

### SUMMARY

The present invention is based on a therapeutic approach for modulating the activity of the human Prkag3 promoter, such that diseases related to energy metabolism, such as obesity, dyslipidemia, insulin resistance syndrome and type 2 diabetes, can be treated or prevented. This approach is based on the hypothesis that AMPK is a major cellular regulator of lipid and glucose metabolism and that modulation of the expression of the AMPK γ3 chain encoded by the Prkag3 gene will be beneficial for treatment of these diseases. The effect of the muscle specific over expression of an AMPK γ3 chain on the glycogen content of skeletal muscle in a transgenic animal model is demonstrated in copending application Ser. No. 60/353,430 (“Transgenic Animals Expressing PRKAG3,” filed Feb. 1, 2002).

In one aspect, the invention features an isolated nucleic acid capable of directing transcription of a heterologous coding sequence positioned downstream therefrom, wherein the nucleic acid is (a) a nucleic acid that includes the nucleotide sequence of nucleotides 1-14970 of SEQ ID NO:1; (b) a functional fragment of the nucleic acid of (a); (c) a nucleic acid that includes a nucleotide sequence functionally equivalent to the nucleic acid of (a) or (b); and (d) a nucleic acid that includes a nucleotide sequence that hybridizes under stringent conditions to a sequence complementary to the nucleic acid of (a), (b), or (c). The nucleic acid can include the nucleotide sequence of nucleotides 1-14970 of SEQ ID NO:1. The nucleic acid of (c) can include a nucleotide sequence that is at least 87% or 95% homologous to nucleotides 1-14970 in SEQ ID NO:1.

In another aspect, the invention features an expression construct effective for directing transcription of a coding sequence, wherein the expression construct includes a nucleic acid capable of directing transcription of a heterologous coding sequence positioned downstream therefrom as described above; and a coding sequence operably linked to the nucleic acid, wherein the coding sequence is heterologous to the nucleic acid. The coding sequence can be a reporter gene such as a reporter gene encoding a reporter molecule selected from the group consisting of beta-galactosidase, beta-glucuronidase, luciferase, chloramphenicol acetyltransferase, neomycin phosphotransferase, and guanine xanthine phosphoribosyltransferase. The expression construct further can include a nucleotide sequence encoding a transactivator protein (e.g., Mothers against decapentaplegic homolog 3 (SMAD3), sterol regulatory element binding protein (SREBP), nuclear factor of activated T-cells (NFAT), upstream stimulating factor (USF), Erythroblastosis virus oncogene homolog 1 (c-Ets-1), acute myelogenous leukemia-1 (AML-1), hepatocyte nuclear factor 3 (HNF3), fork head related activator 2 (FREAC2), fork head related activator 3 (FREAC3), fork head related activator 7 (FREAC7), signal transducer and activator of transcription (STAT), CCAAT/enhancer-binding protein J (CEBP), adaptor protein 1 (AP-1), early growth response factor 1 (Egr-1), early growth response factor 2 (Egr-2), Ets like gene 1 (Elk-1), Myoblast determination protein 1 (MyoD), myocyte specific enhancer binding factor 2 (MEF2), GATA binding factors, α1 feto protein transcription factor (FTF), Reticuloendotheliosis viral oncogene homolog (c-Rel), LIM domain only 2 (Lmo2), hepatic leukemia factor (HLF), myogenic bHLH protein (Myf5), retinoic acid receptor (RAR), Retinoic acid receptor-related orphan receptor (ROR), androgene receptor binding site (ARE), Sp1, or steriogenic factor 1 (SF-1) transactivator proteins).

In another aspect, the invention features host cells that include the expression constructs described above. The host cell can be an immortalized cell. The host cell can be a muscle cell such as ATCC cell lines CRL-1443, CRL-1456, or CRL-2061.

The invention also features a method for expressing a heterologous coding sequence in a host cell. The method includes introducing a first expression construct into the host cell, wherein the first expression construct comprises a nucleic acid described above operably linked to a heterologous coding sequence. The nucleic acid can be identical to the nucleotide sequence represented by nucleotides 1-14970 in SEQ ID NO:1 or a nucleotide sequence functionally equivalent to the nucleic acid sequence represented by nucleotides 1-14970 in SEQ ID NO:1. The expression construct further can include a reporter gene such as a reporter gene encoding a reporter molecule selected from the group consisting of beta-galactosidase, beta-glucuronidase, luciferase, chloramphenicol acetyltransferase, neomycin phosphotransferase, and guanine xanthine phosphoribosyltransferase. The first expression construct can be introduced into the cell by adenovirus infection, liposome-mediated transfer, topical application to the cell, or microinjection. The first expression construct further can include a nucleotide sequence encoding a transactivator protein (e.g., the SMAD3, SREBP, NFAT, USF, c-Ets-1, AML-1, HNF3, FREAC2, FREAC3, FREAC7, STAT, CEBP, AP-1, Egr-1, Egr-2, Elk-1, MyoD, MEF2, GATA, FTF, c-Rel, Lmo2, HLF, Myf5, RAR, ROR, ARE, Sp1, or SF-1 transactivator proteins) or a repressor protein such as a histone deacetylase.

The method further can include introducing a second expression construct into the cell, wherein the second expression construct includes a nucleotide sequence encoding a transactivator protein or repressor protein, or contacting the cell with a transactivator protein or repressor protein. The method further can include contacting the cell with an agonist or antagonist of the transactivator protein or repressor protein.

The invention also features a method of determining whether or not a chemical compound transcriptionally modulates the expression of a Prkag3 gene. The method includes: (a) obtaining a cell line or organism, wherein the cell line or organism comprises an expression construct that contains a reporter gene as described above; (b) contacting the cell line or organism with a chemical compound, and (c) detecting the presence or absence of a detectable signal; wherein the presence or absence of detectable signal is indicative of the transcriptional modulatory activity of the chemical compound. The method further can include (d) quantitatively determining the amount of detectable signal produced in (c); and (e) comparing the amount of signal determined in (d) with the amount of signal detected in the absence of any chemical compound, thereby identifying the chemical compound as a transcriptional modulator of the human Prkag3 promoter. The cell line or organism can include a transactivator protein or a repressor protein. The method further can include contacting the cell line or organism with a transactivator protein or repressor protein.

In yet another aspect, the invention features chemical compounds identified by the above method, and methods of treating or preventing diseases related to energy metabolism in a subject using such compounds. The methods include administering to the subject a therapeutically effective amount of a chemical compound identified using the above methods. The disease related to energy metabolism can be selected from the group consisting of obesity, dyslipidemia, insulin resistance syndrome, and type 2 diabetes.

The invention also features a transgenic non-human mammal (e.g., a mouse) whose germ or somatic cells contain the expression construct described above, and progeny of the transgenic non-human mammal.

In yet another embodiment, the invention features an isolated human Prkag3 gene that includes 14 exons and a promoter, wherein the promoter is selected from the group consisting of: (a) a promoter comprising the nucleotide sequence shown as nucleotides 1-14970 in SEQ ID NO:1; (b) a promoter comprising a nucleotide sequence functionally equivalent to the nucleotide sequence shown as nucleotides 1-14970 in SEQ ID NO:1; and (c) a promoter comprising a nucleotide sequence that hybridizes under stringent conditions to a sequence complementary to the promoter of (a) or (b) in a Southern hybridization reaction.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF THE DRAWING

FIG. 1 is a dot-plot of the homology between the human Prkag3 gene α-axis) and the mouse Prkag3 gene (y-axis).

### DETAILED DESCRIPTION

In general, the invention features a skeletal muscle specific promoter that is capable of directing transcription of a heterologous coding sequence positioned downstream therefrom. As used herein, “skeletal muscle specific” indicates that transcription is primarily in skeletal muscle with minimal transcription in non-skeletal muscle tissues. Modulating the activity of the human Prkag3 promoter provides a therapeutic approach for treating or preventing diseases related to energy metabolism, including obesity, dyslipidemia, insulin resistance syndrome, and type 2 diabetes.

Definitions

As used herein, “isolated” with reference to a promoter refers to a nucleotide sequence corresponding to the regulatory element of a Prkag3 gene, but free of sequences that normally flank one or both sides of the regulatory element in a genome. An isolated promoter can be, for example, a recombinant DNA molecule, provided one of the nucleic acid sequences normally found flanking that recombinant DNA molecule in a naturally-occurring genome is removed or absent. Thus, isolated promoters include, without limitation, a recombinant DNA that exists as a separate molecule (e.g., a genomic DNA fragment produced by PCR or restriction endonuclease treatment) with no flanking sequences present, as well as a recombinant DNA that is incorporated into a vector, an autonomously replicating plasmid, or into the genomic DNA of a plant as part of a hybrid or fusion nucleic acid molecule.

As used herein, “nucleic acid molecule” includes both DNA and RNA and, unless otherwise specified, includes both double-stranded and single-stranded nucleic acids. Also included are hybrids such as DNA-RNA hybrids. Reference to a nucleic acid sequence also can include modified bases as long as the modification does not significantly interfere either with binding of a ligand such as a protein by the nucleic acid or Watson-Crick base pairing.

Two nucleic acid or polypeptide sequences are “substantially homologous” when at least about 80% (e.g., at least about 85%, 90%, 95%, 96%, 97%, 98%, or 99%) of the nucleotides or amino acids are identical over a defined length of the molecule. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (B12seq) program from the stand-alone version of BLASTZ containing BLASTN version 2.0.14 and BLASTP version 2.0.14. This stand-alone version of BLASTZ can be obtained from Fish & Richardson's web site (world wide web at fr.com/blast) or the U.S. government's National Center for Biotechnology Information web site (world wide web at ncbi.nlm.nih.gov). Instructions explaining how to use the B12seq program can be found in the readme file accompanying BLASTZ.

B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. The following command will generate an output file containing a comparison between two sequences: C:\B12seq -i c:\seq1.txt -j c:\seq2.txt -p blastn -o c:\output.txt -q −1-r 2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with sequence from the identified sequence starting with any matched position and ending with any other matched position. A matched position is any position where an identical nucleotide is presented in both the target and identified sequence. Gaps presented in the target sequence are not counted since gaps are not nucleotides. Likewise, gaps presented in the identified sequence are not counted since target sequence nucleotides are counted, not nucleotides from the identified sequence.

The percent identity over a particular length is determined by counting the number of matched positions over that length and dividing that number by the length followed by multiplying the resulting value by 100. For example, if (1) a 1000 nucleotide target sequence is compared to the sequence set forth in SEQ ID NO:1, (2) the B12seq program presents 850 nucleotides from the target sequence aligned with a region of the sequence set forth in SEQ ID NO: 1 where the first and last nucleotides of that 850 nucleotide region are matches, and (3) the number of matches over those 850 aligned nucleotides is 750, then the 1000 nucleotide target sequence contains a length of 850 and a percent identity over that length of 88 (i.e., 750) 850×100 88).

It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

DNA sequences that are substantially homologous can be identified in a Southern hybridization experiment under, for example, stringent conditions. The term “stringent” when used in conjunction with hybridization conditions is as defined in the art, i.e., 15-20° C. under the melting point Tm. Preferably the conditions are highly stringent”, i.e., 5-10° C. under the melting point Tm. High stringency conditions can include the use of low ionic strength buffer and a high temperature for washing, for example, 0.015 M NaCl/0.0015 M sodium citrate (0.1×SSC), 0.1% sodium dodecyl sulfate (SDS) at 65° C. Alternatively, denaturing agents such as formamide can be employed during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Defining appropriate hybridization conditions is within the skill of the art. See, e.g., Molecular Cloning: A Laboratory Manual, 3rd ed., Sambrook et al. eds., Cold Spring Harbor Laboratory Press, 2001; DNA Cloning: A Practical Approach, Glover & Hames eds., Oxford University Press, 1996; Nucleic Acid Hybridization: Essential Techniques, Ross ed., Wiley, 1998.

A sequence “functionally equivalent” to a Prkag3 promoter sequence is one which functions in a similar manner as the Prkag3 promoter sequence. Thus, a promoter sequence “functionally equivalent” to the Prkag3 promoter described herein is one which is capable of directing transcription of a downstream coding sequence in similar time frames of expression, in similar amounts, and with similar tissue specificity as the Prkag3 promoter.

A “functional fragment” of a Prkag3 promoter sequence is a fragment that functions in a similar manner as the Prkag3 promoter sequence. Thus, a fragment that is a “functional fragment” of a Prkag3 promoter described herein is a nucleic acid fragment that is capable of directing transcription of a downstream coding sequence in similar time frames of expression, in similar amounts, and with similar tissue specificity as the Prkag3 promoter.

A DNA “coding sequence” or a “nucleotide sequence encoding” a particular protein is a nucleic acid sequence which can be transcribed and translated into a polypeptide in vivo or in vitro when placed under the control of appropriate regulatory sequences. The boundaries of the coding sequence are determined by a start codon at the 5′-(amino) terminus and a translation stop codon at the 3′-(carboxy) terminus. A coding sequence can include, but is not limited to, prokaryotic sequences, cDNAs from eukaryotic mRNA, genomic DNA sequences from eukaryotic (e.g., mammalian) sources, viral RNA or DNA, and even synthetic nucleotide sequences.

DNA “control sequences” refers collectively to promoter sequences, polyadenylation signals, transcription termination sequences, upstream regulatory domains, enhancers, untranslated regions, including 5′-UTRs and 3′-UTRs, which collectively provide for the transcription and translation of a coding sequence in a host cell. A transcription termination sequence will usually be located 3′ to the coding sequence.

As used herein, “operably linked” refers to a connection between a promoter and/or other control elements to a coding sequence in such a way as to permit expression of the coding sequence. The control sequences need not be contiguous with a coding sequence, so long as they function to direct the expression thereof. Thus, for example, intervening untranslated yet transcribed sequences can be present between a promoter and the coding sequence and the promoter can still be considered “operably linked” to the coding sequence.

A control sequence “directs the transcription” of a coding sequence in a cell when RNA polymerase will bind the promoter sequence and transcribe the coding sequence into mRNA, which can then be translated into a polypeptide.

A cell has been “transformed” by exogenous DNA when such exogenous DNA has been introduced inside the cell membrane. Exogenous DNA may or may not be integrated (covalently linked) into chromosomal DNA making up the genome of the cell. In eukaryotic cells, a stably transformed cell is generally one in which the exogenous DNA has become integrated into the chromosome so that it is inherited by daughter cells through chromosome replication, or one which includes stably maintained extra chromosomal plasmids. This stability is demonstrated by the ability of the eukaryotic cell to establish cell lines or clones comprised of a population of daughter cells containing the exogenous DNA.

A “heterologous” nucleic acid is an identifiable segment of nucleic acid within or attached to another nucleic acid molecule that is not found in association with the other molecule in nature. For example, a sequence encoding a protein other than Prkag3 is considered a heterologous sequence when linked to a Prkag3 promoter. Similarly, a sequence encoding a Prkag3 gene will be considered heterologous when linked to a Prkag3 gene promoter with which it is not normally associated. Another example of a heterologous coding sequence is a construct where the coding sequence itself is not found in nature (e.g., synthetic sequences having codons different from the native gene). Likewise, a chimeric sequence, that includes, for example, a heterologous structural gene and a gene encoding a Prkag3 protein or a portion of a Prkag3 protein, linked to a Prkag3 promoter, whether derived from the same or a different organism, will be considered heterologous since such chimeric constructs are not normally found in nature. Allelic variation or naturally occurring mutational events do not give rise to a heterologous region of DNA, as used herein.

As used herein, “transcriptional modulator” refers to a molecule (e.g., a polypeptide) which effects activity of a promoter or other control sequence by either (a) direct binding of the molecule to promoter sequences, a DNA- or RNA-binding protein, and/or a DNA- or RNA-binding protein complex, or (b) direct binding of the molecule to a protein which directly chemically modifies a DNA- or RNA-binding protein or protein complex.

The phrase “specifically transcriptionally modulate expression” as used herein means modulating the activity of a Prkag3 promoter without modulating the activity of other promoters in the cell in a way which would cause an adverse effect on (a) an organism containing the cell in the case where the cell is within the organism or (b) the growth or the culturing of the cell, in the case where the cell is being grown or cultured.

Promoters

In one embodiment, the invention features a nucleic acid having the nucleotide sequence shown as nucleotides 1-14970 in SEQ ID NO:1. Such a nucleic acid is a promoter, i.e., is capable of directing transcription of a heterologous coding sequence positioned downstream therefrom. Functional fragments of the promoter can be made that retain the ability to promote expression of a nucleic acid molecule of interest. In general, fragments of the promoter are at least 30 nucleotides in length, e.g., about 50, 100, 200, 400, 600, or 800 nucleotides in length. For example, a functional fragment of the human Prkag3 promoter can include one or more of the fragments corresponding to nucleotides 39-537, 2386-2406, 2099-2591, 3585-3604, 3817-3869, 5172-5206, 5387-5427, 5595-5623, 5713-5747, 5752-5772, 8028-8081, 8568-8588, 8892-8909, 9151-9216, 9883-9932, 10360-10389, 11242-11269, 11921-11977, 12128-12177, 12170-12209, 12244-12276, 12267-12333, 12717-12766, 13258-13298, 13302-13371, 13726-13807, 13986-14136, 14139-14162, 14146-14169, 14146-14175, 14709-14758, and 14849-14877 of the nucleotide sequence shown in SEQ ID NO:1. Fragments containing nucleotides 11000 to 14970 are particularly useful as this region is conserved among humans, rats, and mice.

The ability of fragments to promote expression of a nucleic acid molecule can be assayed using the methods described herein. In particular, the fragment can be operably linked to a nucleic acid sequence and used to transiently or stably transform a eukaryotic cell (e.g., a skeletal muscle cell line). Expression of the gene product encoded by the nucleic acid sequence can be monitored in such a transformed cell using standard techniques. Promoter fragments also can be used as hybridization probes.

A promoter of the invention can have a nucleotide sequence that is functionally equivalent to the nucleotide sequence shown as nucleotides 1-14970 in SEQ ID NO:1 or a functional fragment thereof, and retain the ability to promote expression of a heterologous nucleic acid molecule. For example, the promoter can be a nucleic acid molecule having a nucleotide sequence that is at least 85% identical to nucleotides 1-14970 or nucleotides 11000-14970 of SEQ ID NO: 1 (e.g., at least 87%, 90%, 95%, or 99% identical to nucleotides 11000-14970 or 1-14970 of SEQ ID NO:1).

A promoter also can be a nucleic acid molecule that includes a nucleotide sequence that hybridizes under stringent conditions to a sequence complementary to the following: a nucleic acid having the nucleotide sequence shown as nucleotides 1-14970 in SEQ ID NO: 1, a functional fragment of the nucleotide sequence of nucleotides 1-14970 in SEQ ID NO: 1, or a nucleic acid having a nucleotide sequence functionally equivalent to the nucleotide sequence of nucleotides 1-14970 in SEQ ID NO:1.

Expression Constructs

This invention also provides an expression construct effective in directing the transcription of a selected coding sequence. Suitable expression constructs include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Suitable expression constructs may include elements from more than one virus. Retrovirus or adenovirus based vectors are particularly useful for eukaryotic cells. Such vectors may include all or a part of a viral genome, such as long term repeats (LTRs), promoters (e.g., CMV promoters, SV40 promoters, RSV promoter), enhancers, and so forth. For prokaryotic cells, phage based vectors (e.g., lambda phage) are particular useful.

Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

Typically, an expression construct includes a Prkag3 promoter described above and a coding sequence operably linked to the promoter, wherein the coding sequence is heterologous to the promoter. The coding sequence can be a reporter gene, such as a reporter gene encoding a reporter molecule such as beta-galactosidase, beta-glucuronidase, luciferase, chloramphenicol acetyltransferase, neomycin phosphotransferase, and guaninexanthine phosphoribosyltransferase. Fluorescent proteins such as GFP (green fluorescent protein) and YFP (yellow fluorescent protein) also are useful. Such reporter molecules allow expression to be monitored easily.

The expression construct may further include a nucleotide sequence encoding a transactivator protein capable of modulating the activity of the Prkag3 promoter. The transactivator protein may be the SMAD3, SREBP, NFAT, USF, c-Ets-1, AML-1, HNF3, FREAC2, FREAC3, FREAC7, STAT, CEBP, AP-1, Egr-1, Egr-2, Elk-1, MyoD, MEF2, GATA, FTF, c-Rel, Lmo2, HLF, Myf5, RAR, ROR, ARE, Sp1, or SF-1 transactivator proteins. The nucleotide sequence encoding the transactivator protein can be operably linked to a constitutively active promoter (e.g., a viral promoter).

The recombinant expression construct may further include a nucleotide sequence encoding a repressor protein capable of modulating the activity of the Prkag3 promoter. The repressor protein may be a histone deacetylase (HDAC), MEF2-interacting transcription repressor (MITR), silencing mediator for retinoid and thyroid hormone receptors (SMRT), nuclear corepressor (N-CoR), Small Unique Nuclear receptor CoRepressor (SUN-CoR), TG interacting factor (TGIF), Sloan Kettering virus oncogene homolog (Ski), Ski-related novel gene (Sno), NGFI-A-binding protein (NAB), or Friend of GATA (FOG). The nucleotide sequence encoding a repressor can be operably linked to a constitutively active promoter such as a viral promoter.

Host Cells

This invention also provides host cells that include the expression constructs discussed above. Preferably, the host cell is a cell expressing a native Prkag3 gene, such as a muscle cell. Suitable muscle cell lines are available from the American Type Culture Collection (ATCC), including the ATCC cell lines CRL-1443, CRL-1456, and CRL-2061.

Host cells can be transiently transfected, which indicates that the exogenous nucleic acid is episomal (i.e., not integrated into the chromosomal DNA). In other embodiments, the cells are stably transfected, i.e., the exogenous nucleic acid is integrated into the host cell's chromosomal DNA. As used herein, “transformed” and “transfected” encompass the introduction of a nucleic acid molecule (e.g., an expression construct) into a cell by one of a number of techniques that are well established within the art. Prokaryotic cells can be transformed with nucleic acids by, for example, electroporation or calcium chloride mediated transformation. Nucleic acids can be transfected into eukaryotic cells by techniques including, for example, calcium phosphate co-precipitation, DEAE-dextran-mediated transfection, lipofection, electroporation, or microinjection. Suitable methods for transforming and transfecting host cells are found in Sambrook et al., Molecular Cloning: A Laboratory Manual (2nd edition), Cold Spring Harbor Laboratory, New York (1989), and reagents for transformation and/or transfection are commercially available (e.g., Lipofectin (Invitrogen/Life Technologies); Fugene (Roche, Indianapolis, Ind.); and SuperFect (Qiagen, Valencia, Calif.)).

For example, an expression construct that includes a nucleotide sequence encoding a transactivator protein capable of activating the Prkag3 promoter can be introduced into a host cell. The transactivator protein may be the SMAD3, SREBP, NFAT, USF, c-Ets-1, AML-1, HNF3, FREAC2, FREAC3, FREAC7, STAT, CEBP, AP-1, Egr-1, Egr-2, Elk-1, MyoD, MEF2, GATA, FTF, c-Rel, Lmo2, HLF, Myf5, RAR, ROR, ARE, Sp 1, or SF-1 transactivator proteins. In one embodiment, the expression construct may include a nucleic acid segment encoding a repressor protein capable of repressing the Prkag3 promoter. The repressor protein may be a histone deacetylase (HDAC).

In another embodiment, the host cell can be contacted with a transactivator protein or a repressor protein capable of modulating the Prkag3 promoter. Suitable transactivator proteins and repressor proteins are described above. The method may also include contacting the cell with an agonist or antagonist of the transactivator protein or the repressor protein.

Methods of Identifying Modulaters of the Prkag3 Promoter

The invention also provides a method of determining whether or not a chemical compound transcriptionally modulates the expression of a Prkag3 gene, (e.g., the human Prkag3 gene). Such a method can include obtaining a cell line or organism, wherein the cell line or organism includes an expression construct comprising a Prkag3 promoter described above operably linked to a reporter gene; and contacting the cell line or organism with a chemical compound under conditions appropriate for transcription to occur. Generally, the presence or absence of detectable signal is indicative of the transcriptional modulatory activity of the chemical compound. The method further can include quantitatively determining the amount of the signal produced and comparing the amount of signal determined with the amount of signal detected in the absence of any chemical compound or with the amount of signal produced and detected upon contacting the cell line or organism with other chemical compounds. Using this method, a chemical compound can be identified that transcriptionally modulates expression of the human Prkag3 gene.

In the method, the cell line or organism may express a transactivator protein or repressor protein capable of modulating the activity of the Prkag3 promoter. Alternatively, the method may include contacting the cell line or organism with a transactivator protein or a repressor protein capable of modulating the Prkag3 promoter.

In the method, the contacting may be effected from about 1 hour to about 24 hours. The contacting may be effected with more than one predetermined concentration of the chemical compound to be tested. The chemical compound to be tested may be a polypeptide of at least 2 amino acids, e.g., 2 to 6 amino acids, 7 to 12 amino acids, 2 to 20 amino acids, or greater than 20 amino acids, such as 50 or more amino acids. For drug screening purposes, preferred compounds are chemical compounds of low molecular weight and potential therapeutic agents. For example, the compounds can have a molecular weight of less than about 1000 Daltons, such as less than 800, 600 or 400 Daltons in weight. If desired, the chemical compound may be a member of a chemical library. The library may comprise any number of individual members, for example, tens to hundreds to thousands to millions etc., of suitable compounds. Representative compounds include, but are not limited to, peptides, peptoids and other oligomeric compounds (cyclic or linear), and template-based smaller molecules. For example, the compounds can be benzodiazepines, hydantoins, biaryls, carbocyclic and polycyclic compounds (e.g., naphthalenes, phenothiazines, acridines, steroids etc.), carbohydrate and amino acids derivatives, dihydropyridines, benzhydryls and heterocycles (e.g., triazines, indoles, thiazolidines etc.). Preferred chemical libraries include chemical compounds of low molecular weight and potential therapeutic agents.

Preferably, the chemical compound to be tested is a chemical compound not previously known to be a modulator of the human Prkag3 gene.

In another embodiment, the present invention provides use of a chemical compound able to modulate the expression of the human Prkag3 gene in preparation of a medicament for the treatment or prevention of diseases related to energy metabolism, such as obesity, dyslipidemia, insulin resistance syndrome, and type 2 diabetes. Preferably the compound is an activator of the expression of the human Prkag3 gene.

This invention also provides a method of treating or preventing diseases related to energy metabolism, such as obesity, dyslipidemia, insulin resistance syndrome and type 2 diabetes, in a subject which comprises administering to the subject a therapeutically effective amount of a chemical compound identified by the method of the invention.

Transgenic Animals

The invention also provides a transgenic non-human mammal (and progeny therefrom) whose germ or somatic cells contain the expression construct described herein. Non-human mammals include, for example, rodents such as rats, guinea pigs, and mice, and farm animals such as pigs, sheep, goats, horses, and cattle. The mammal may be a mouse.

Non-human mammals of the invention such as mice can be used, for example, to identify modulators of the Prkag3 promoter. For example, modulators can be assessed in a first group of such non-human mammals in the presence of a compound, and compared with activity or toxicity in a corresponding control group in the absence of the compound. Suitable compounds are described above. The concentration of compound to be tested depends on the type of compound and in vitro test data.

Non-human mammals can be exposed to test compounds by any route of administration, including enterally (e.g., orally) and parenterally (e.g., subcutaneously, intravascularly, intramuscularly, or intranasally). Suitable formulations for oral administration can include tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). Tablets can be coated by methods known in the art. Preparations for oral administration can also be formulated to give controlled release of the compound.

Compounds can be prepared for parenteral administration in liquid form (e.g., solutions, solvents, suspensions, and emulsions) including sterile aqueous or non-aqueous carriers. Aqueous carriers include, without limitation, water, alcohol, saline, and buffered solutions. Examples of non-aqueous carriers include, without limitation, propylene glycol, polyethylene glycol, vegetable oils, and injectable organic esters. Preservatives and other additives such as, for example, antimicrobials, anti-oxidants, chelating agents, inert gases, and the like may also be present. Pharmaceutically acceptable carriers for intravenous administration include solutions containing pharmaceutically acceptable salts or sugars. Intranasal preparations can be presented in a liquid form (e.g., nasal drops or aerosols) or as a dry product (e.g., a powder). Both liquid and dry nasal preparations can be administered using a suitable inhalation device. Nebulised aqueous suspensions or solutions can also be prepared with or without a suitable pH and/or tonicity adjustment.

### EXAMPLES

Identification and Characterization of the Prkag3 Promoter

The published cDNA sequences encoding the human AMPK γ3 subunit (Genbank Accession Nos. AJ249977 and AF214519) were used the search the database for genomic sequences comprising the human Prkag3 gene and promoter. The human BAC clone RP11-459119 (Genbank accession no AC009974) was identified and found to comprise the complete Prkag3 gene (SEQ ID NO: 1). The coding part of the gene was found to comprise at least 14 exons and spanned more than 8 kb. The 5′ end of the reported cDNA sequence (AJ249977) consists of a donor-acceptor splice signal indicating the possible presence of yet another exon in the 5′ end of the gene.

Identification of Specific Promoter Elements

The sequence upstream of the suggested ATG initiation codon was analysed for the presence of potential promoter elements using the Model Inspector algorithm (release 4.8) (Frech K. et al., 1997, J Mol. Biol., 270: 674-687) at the Genomatix Software Internet site (world wide web at genomatix.de). Identified potential promoter elements are presented in Table 1.

TABLE 1
Potential promoter elements identified in a 15 kb
fragment of the 5′ upstream region the human Prkag3 gene.

| Positions in SEQ | |
|---|---|
| ID NO: 1 | Promoter element |
| 39-537 | SMAD_EBOX_02 |
| 2386-2406 | SORY_NFAT_01 |
| 2099-2591 | SMAD_EBOX_02 |
| 3585-3604 | ETSF_HAML_01 |
| 3817-3869 | FKHD_FKHD_01 |
| 5172-5206 | CEBP_STAT_01 |
| 5387-5427 | GATA_AP1F_02 |
| 5595-5623 | SF1F_EGRF_01 |
| 5713-5747 | EGRF_NFAT_01 |
| 5752-5772 | SP1F_ETSF_01 |
| 8028-8081 | FKHD_FKHD_01 |
| 8568-8588 | CEBP_STAT_01 |
| 8892-8909 | MEF2_MEF2 |
| 9151-9216 | SOX9_SF1F_01 |
| 9883-9932 | Promoter/Transcription start at 9923 |
| 10360-10389 | NFAT_AP1F_02 |
| 11242-11269 | CEBP_NFKB_0 |
| 11921-11977 | MYOD_TBPF_01 |
| 12128-12177 | Promoter/Transcription start at 12168 |
| 12170-12209 | Promoter/Transcription start at 12199 |
| 12244-12276 | SF1F_CREB_01 |
| 12267-12333 | SOX9_SF1F_01 |
| 12717-12766 | Promoter/Transcription start at 12757 |
| 13258-13298 | MYOD_MYOD_01 |
| 13302-13371 | RORA_GATA_01 |
| 13726-13807 | GREF_FKHD_01 |
| 13986-14136 | NFAT_AP1F_01 |
| 14139-14162 | ETSF_AP1F_01 |
| 14146-14169 | ETSF_AP1F_01 |
| 14146-14175 | NFKB_AP1F_01 |
| 14709-14758 | Promoter/Transcription start at 14749 |
| 14849-14877 | SP1F_CEBP_01 |

The identified potential promoter elements listed in Table 1 were further analysed for the presence of potential transcription factor binding sites by comparison with matrices in the TRANSFAC database (Wingender E. et al., 2000, Nucleic Acids Res., 28: 316-319). The identified transcription factor binding sites and the corresponding transcription factors or motifs are listed in Table 2.

TABLE 2
Potential transcription factor binding sites identified in
a 15 kb fragment of the 5′ upstream region the human Prkag3 gene.

| Positions in SEQ | |
|---|---|
| ID NO: 1 | Transcription factor/motif |
| 42-47 | SMAD3 |
| 524-533 | SREBP, sterol regulatory element binding protein |
| 2389-2402 | SRY-related HMG box |
| 2398-2404 | NFAT, nuclear factor of activated T-cells |
| 2583-2588 | SMAD3 |
| 2105-2110 | USF, upstream stimulating factor |
| 3589-3597 | c-Ets-1 binding site |
| 3594-3599 | AML-1, Acute myelogenous leukemia-1 |
| 3820-3830 | HNF3, hepatocyte nuclear factor 3 |
| 3859-3866 | FREAC2, fork head related activator 2 |
| 5177-5185 | STAT, signal transducer and activator of transcription |
| 5189-5200 | CEBP, CCAAT/enhancer-binding protein β |
| 5389-5394 | GATA |
| 5417-5425 | AP-1, adaptor protein 1, Fos-Jun dimer |
| 5595-5606 | Egr-1, early growth response factor 1 |
| 5609-5615 | RAR, retinoic acid receptor |
| 5615-5622 | SF1, stereogenic factor 1 |
| 5716-5726 | Egr-2, early growth response factor 2 |
| 5739-5745 | NFAT, nuclear factor of activated T-cells |
| 5756-5763 | Elk-1 |
| 5759-5771 | GC box |
| 8029-8039 | FREAC3, fork head related activator 3 |
| 8068-8076 | FREAC7, fork head related activator 7 |
| 8571-8581 | CEBP, CCAAT/enhancer-binding protein β |
| 8580-8588 | STAT, signal transducer and activator of transcription |
| 8896-8905 | MEF2, myocyte specific enhancer binding factor 2 |
| 9157-9164 | SRY-related HMG box |
| 9206-9213 | FTF, α1-feto protein transcription factor |
| 10362-10368 | NFAT, nuclear factor of activated T-cells |
| 10372-10380 | AP-1, adaptor protein 1, Fos-Jun dimer |
| 11249-11259 | CEBP, CCAAT/enhancer-binding protein β |
| 11258-11267 | c-Rel |
| 11925-11930 | Lmo2 complex |
| 11963-11971 | TATA |
| 12249-12258 | HLF, hepatic leukemia factor |
| 12267-12274 | FTF, α1-feto protein transcription factor |
| 12273-12280 | SRY-related HMG box |
| 12323-12330 | FTF, α1-feto protein transcription factor |
| 12717-12766 | Promoter/Transcription start at 12757 |
| 13263-13268 | Lmo2 complex |
| 13286-13297 | Myf5, myogenic bHLH protein |
| 13306-13311 | GATA |
| 13360-13370 | ROR, RAR related orphan receptor |
| 13732-13746 | ARE, androgene receptor binding site |
| 13796-13806 | HNF3, hepatocyte nuclear factor 3 |
| 13988-13994 | NFAT, nuclear factor of activated T-cells |
| 14125-14133 | AP1, activator protein 1 |
| 14143-14151 | c-Ets-1 binding site |
| 14149-14157 | AP1, activator protein |
| 14157-14164 | c-Ets-1 binding site |
| 14163-14172 | c-Rel |
| 14850-14862 | GC box |
| 14865-14876 | CEBP, CCAAT/enhancer-binding protein β |

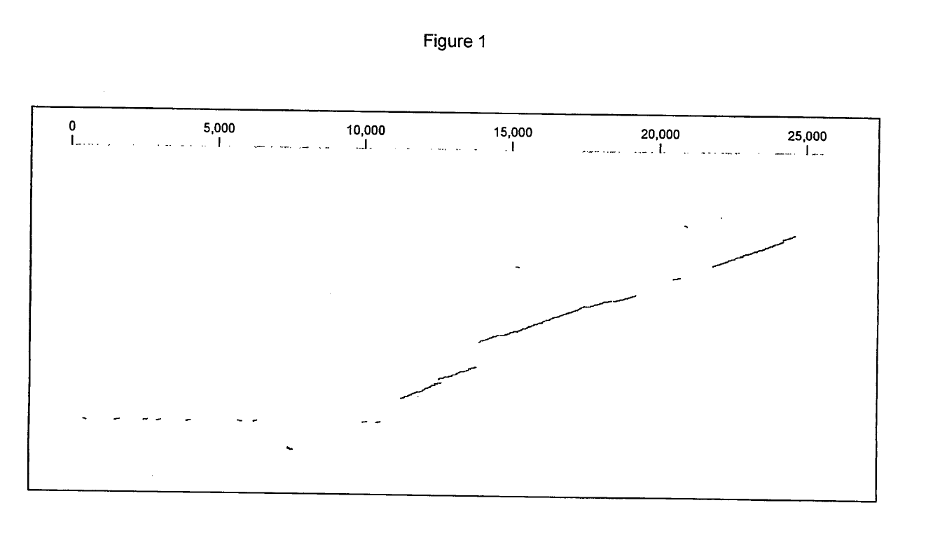

An alignment between the 26 kb human DNA sequence (SEQ ID NO:1) and an orthologous 30 kb mouse genomic DNA sequence (Chromosome 1, bp 75450000-75480000, Ensembl Jan. 7, 2003) comprising the mouse Prkag3 gene was done with the alignment program BlastZ (world wide web at bio.cse.psu.edu/) (Schwartz S. et al., 2000. Genome Res., 10: 577-86) with the default settings. The interactive alignment viewer Laj (local alignments with java) (world wide web at bio.cse.psu.edu) was used to visualize the output from BlastZ. The resulting dot-plot is presented as FIG. 1 with the human sequence along the x-axis and the mouse sequence along the y-axis. In FIG. 1, the threshold is 50% homology to create a “dot” in the plot. The actual homology varies between 50 and 80% in the conserved promoter regions (and the introns) compared to >90% homology in the exons. Regions of higher homology (i.e., conserved sequences) indicate that the region may be of function.

The most prominent homology between the sequences can be seen from bp 11000-26000 of the human sequence (SEQ ID NO:1). This partofthe SEQ ID NO:1 comprises the transcribed part of the Prkag3 gene and 4 kb of 5′ upstream sequence. This indicates that transcription factor binding sites that are conserved between mouse and human can be found between bp 11000 and 15000 of SEQ ID NO: 1. Similar results were obtained by comparison of SEQ ID NO:1 with an orthologous rat genomic DNA sequence.

Expression Constructs

Prkag3 promoter fragments are generated by PCR amplification using genomic DNA comprising the Prkag3 gene, e.g., the BAC RP11-459I19, as template. Promoter fragments are subsequently cloned into a reporter vector comprising a suitable reporter gene in such a manner that the expression of the reporter gene is directed by the promoter fragment. An example of such a reporter vector is the pGL3-Basic luciferase reporter vector (Promega, Madison, Mich.).

Reporter Assay

Expression constructs are introduced into mammalian cells expressing a native Prkag3 gene as evident by the production of AMPK containing a γ3 subunit. Suitable cells are e.g., muscle cells. Expression of the reporter gene is measured using a suitable assay, e.g., in the case of luciferase as the reporter, the Luciferase Assay System (Promega, Madison, Mich.). The level of expression of the reporter gene is taken as a measure of the activity of the promoter fragment.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated nucleic acid capable of directing transcription of a heterologous coding sequence positioned downstream therefrom, wherein the nucleic acid is selected from the group consisting of:
(a) a nucleic acid comprising the nucleotide sequence of nucleotides 1-14970 of SEQ ID NO:1;
(b) a functional fragment of the nucleic acid of (a);
(c) a nucleic acid comprising a nucleotide sequence functionally equivalent to the nucleic acid of (a) or (b); and
(d) a nucleic acid comprising a nucleotide sequence that hybridizes under stringent conditions to a sequence complementary to the nucleic acid of (a), (b), or (c).

2. The nucleic acid of claim 1, wherein the nucleic acid comprises the nucleotide sequence of nucleotides 1-14970 of SEQ ID NO:1.

3. The nucleic acid of claim 1, wherein the nucleic acid of (c) comprises a nucleotide sequence that is at least 87% homologous to nucleotides 1-14970 in SEQ ID NO:1.

4. The nucleic acid of claim 3, wherein the nucleic acid of (c) comprises a nucleotide sequence that is at least 95% homologous to nucleotides 1-14970 in SEQ ID NO:1.

5. An expression construct effective for directing transcription of a coding sequence, wherein the expression construct comprises:
(a) a nucleic acid according to claim 1; and
(b) a coding sequence operably linked to the nucleic acid, wherein the coding sequence is heterologous to the nucleic acid.

6. The expression construct of claim 5, wherein the coding sequence is a reporter gene.

7. The expression construct of claim 6, wherein the reporter gene encodes a reporter molecule selected from the group consisting of beta-galactosidase, beta-glucuronidase, luciferase, chloramphenicol acetyltransferase, neomycin phosphotransferase, and guanine xanthine phosphoribosyltransferase.

8. The expression construct of claim 5, further comprising a nucleotide sequence encoding a transactivator protein.

9. The expression construct of claim 8, wherein the transactivator protein is selected from the group consisting of the SMAD3, SREBP, NFAT, USF, c-Ets-1, AML-1, HNF3, FREAC2, FREAC3, FREAC7, STAT, CEBP, AP-1, Egr-1, Egr-2, Elk-1, MyoD, MEF2, GATA, FTF, c-Rel, Lmo2, HLF, Myf5, RAR, ROR, ARE, Sp1, and SF-1 transactivator proteins.

10. A host cell comprising the expression construct according to claim 5.

11. The host cell of claim 10, wherein the host cell is an immortalized cell.

12. The host cell of claim 10, wherein the host cell is a muscle cell.

13. The host cell of claim 12, wherein the muscle cell is selected from the group consisting of the ATCC cell lines CRL-1443, CRL-1456, and CRL-2061.

14. A method for expressing a heterologous coding sequence in a host cell comprising: introducing a first expression construct into the host cell, wherein the first expression construct comprises a nucleic acid according to claim 1 operably linked to a heterologous coding sequence.

15. The method of claim 14, wherein the nucleic acid is identical to the nucleotide sequence represented by nucleotides 1-14970 in SEQ ID NO:1.

16. The method of claim 14, wherein the nucleic acid is a nucleotide sequence functionally equivalent to the nucleic acid sequence represented by nucleotides 1-14970 in SEQ ID NO:1.

17. The method of claim 14, wherein the expression construct further comprises a reporter gene.

18. The method of claim 17, wherein the reporter gene encodes a reporter molecule selected from the group consisting of beta-galactosidase, beta-glucuronidase, luciferase, chloramphenicol acetyltransferase, neomycin phosphotransferase, and guanine xanthine phosphoribosyltransferase.

19. The method of claim 14, wherein the first expression construct is introduced into the cell by adenovirus infection, liposome-mediated transfer, topical application to the cell, or microinjection.

20. The method of claim 14, wherein the first expression construct further comprises a nucleotide sequence encoding a transactivator protein.

21. The method of claim 20, wherein the transactivator protein is selected from the group consisting of the SMAD3, SREBP, NFAT, USF, c-Ets-1, AML-1, HNF3, FREAC2, FREAC3, FREAC7, STAT, CEBP, AP-1, Egr-1, Egr-2, Elk-1, MyoD, MEF2, GATA, FTF, c-Rel, Lmo2, HLF, Myf5, RAR, ROR, ARE, Sp1, and SF-1 transactivator proteins.

22. The method of claim 14, further comprising introducing a second expression construct into the cell, wherein the second expression construct comprises a nucleotide sequence encoding a transactivator protein.

23. The method of claim 22, wherein the transactivator protein is selected from the group consisting of the SMAD3, SREBP, NFAT, USF, c-Ets-1, AML-1, HNF3, FREAC2, FREAC3, FREAC7, STAT, CEBP, AP-1, Egr-1, Egr-2, Elk-1, MyoD, MEF2, GATA, FTF, c-Rel, Lmo2, HLF, Myf5, RAR, ROR, ARE, Sp1, and SF-1 transactivator proteins.

24. The method of claim 14, further comprising contacting the cell with a transactivator protein.

25. The method of claim 24, wherein the transactivator protein is selected from the group consisting of the SMAD3, SREBP, NFAT, USF, c-Ets-1, AML-1, HNF3, FREAC2, FREAC3, FREAC7, STAT, CEBP, AP-1, Egr-1, Egr-2, Elk-1, MyoD, MEF2, GATA, FTF, c-Rel, Lmo2, HLF, Myf5, RAR, ROR, ARE, Sp1, and SF-1 transactivator proteins.

26. The method of claim 20, further comprising contacting the cell with an agonist or antagonist of the transactivator protein.

27. The method of claim 14, wherein the first expression construct further comprises a nucleotide sequence encoding a repressor protein.

28. The method of claim 27, wherein the repressor protein is selected from the group consisting of a histone deacetylase, MITR, SMRT, N-CoR, SUN-CoR, TGIF, Ski, Sno, NAB, and FOG repressor proteins.

29. The method of claim 14, further comprising introducing a second expression construct into the cell, wherein the second expression construct comprises a nucleotide sequence encoding a repressor protein.

30. The method of claim 29, wherein the repressor protein is selected from the group consisting of a histone deacetylase, MITR, SMRT, N-CoR, SUN-CoR, TGIF, Ski, Sno, NAB, and FOG repressor proteins.

31. The method of claim 14, further comprising contacting the cell with a repressor protein.

32. The method of claim 31, wherein the repressor protein is selected from the group consisting of a histone deacetylase, MITR, SMRT, N-CoR, SUN-CoR, TGIF, Ski, Sno, NAB, and FOG repressor proteins.

33. The method of claim 27, further comprising contacting the cell with an agonist or antagonist of the repressor protein.

34. A method of determining whether or not a chemical compound transcriptionally modulates the expression of a Prkag3 gene, wherein the method comprises:
(a) obtaining a cell line or organism, wherein the cell line or organism comprises the expression construct of claim 6,
(b) contacting the cell line or organism with a chemical compound, and
(c) detecting the presence or absence of a detectable signal; wherein the presence or absence of detectable signal is indicative of the transcriptional modulatory activity of the chemical compound.

35. The method of claim 34, further comprising:
(d) quantitatively determining the amount of detectable signal produced in (c); and
(e) comparing the amount of signal determined in (d) with the amount of signal detected in the absence of any chemical compound, thereby identifying the chemical compound as a transcriptional modulator of the human Prkag3 promoter.

36. The method of claim 34, wherein the cell line or organism comprises a transactivator protein.

37. The method of claim 36, wherein the transactivator protein is selected from the group consisting of the SMAD3, SREBP, NFAT, USF, c-Ets-1, AML1, HNF3, FREAC2, FREAC3, FREAC7, STAT, CEBP, AP-1, Egr-1, Egr-2, Elk-1, MyoD, MEF2, GATA, FTF, c-Rel, Lmo2, HLF, Myf5, RAR, ROR, ARE, Sp1, and SF-1 transactivator proteins.

38. The method of claim 34, further comprising contacting the cell line or organism with a transactivator protein.

39. The method of claim 38, wherein the transactivator protein is selected from the group consisting of the SMAD3, SREBP, NFAT, USF, c-Ets-1, AML-1, HNF3, FREAC2, FREAC3, FREAC7, STAT, CEBP, AP-1, Egr-1, Egr-2, Elk-1, MyoD, MEF2, GATA, FTF, c-Rel, Lmo2, HLF, Myf5, RAR, ROR, ARE, Sp1, and SF-1 transactivator proteins.

40. The method of claim 34, wherein the cell line or organism comprises a repressor protein.

41. The method of claim 40, wherein the repressor protein is a histone deacetylase.

42. The method of claim 34, further comprising contacting the cell line or organism with a repressor protein.

43. The method of claim 42, wherein the transactivator protein is selected from the group consisting of SMAD3, SREBP, NFAT, USF, c-Ets-1, AML-1, HNF3, FREAC2, FREAC3, FREAC7, STAT, CEBP, AP-1, Egr-1, Egr-2, Elk-1, MyoD, MEF2, GATA, FTF, c-Rel, Lmo2, HLF, Myf5, RAR, ROR, ARE, Sp1, and SF-1 transactivator proteins.

44. The method of claim 34, wherein the reporter gene encodes a reporter molecule selected from the group consisting of luciferase, chloramphenicol acetyltransferase, beta-glucuronidase, beta-galactosidase, neomycin phosphotransferase, or guanine xanthine phosphoribosyltransferase.

45. A method of treating or preventing diseases related to energy metabolism in a subject, the method comprising administering to the subject a therapeutically effective amount of a chemical compound identified by the method of claim 31.

46. The method of claim 45, wherein the disease related to energy metabolism is selected from the group consisting of obesity, dyslipidemia, insulin resistance syndrome, and type 2 diabetes.

47. A transgenic non-human mammal whose germ or somatic cells contain the expression construct of claim 5.

48. Progeny of the transgenic non-human mammal of claim 47.

49. The transgenic non-human mammal of claim 47, wherein the mammal is a mouse.

50. A composition comprising a chemical compound identified by the method of claim 34.

51. An isolated human Prkag3 gene comprising 14 exons and a promoter, wherein the promoter is selected from the group consisting of:
(a) a promoter comprising the nucleotide sequence shown as nucleotides 1-14970 in SEQ ID NO:1;
(b) a promoter comprising a nucleotide sequence functionally equivalent to the nucleotide sequence shown as nucleotides 1-14970 in SEQ ID NO:1; and
(c) a promoter comprising a nucleotide sequence that hybridizes under stringent conditions to a sequence complementary to the promoter of (a) or (b) in a Southern hybridization reaction.


SEQUENCE LISTING (2 sequences)

SEQ ID NO: 1 - DNA, 26000 bp, Homo sapiens
  protein_bind (42)..(47): SMAD3
cccagatggggtggctgccgggcggagaggctcctcacttctcagacggggcagctgccgggcggaggggctcctcacttctcagacggggcggttgccaggcagagggtctcctcacttctcagacggggcagccgggcagagacgctcctcacctcccagacggggtctcggccgggcagaggcgctcctcacatcccagatggggtggcggggcagaggcactccccacatctcagacgatgggcggccgggcagagacgctcctcacttcctagatgtgatggcggctgggaagaggcgctcctcacttcctagatgggatggcggccgggtggagacgctcctcactttccagactgggcagccaggcagaggggctcctcacatcccagatgatgggcggccaggcagagacactcctcacttcccagacggggtggcggccgggcagaggctgcaatctcggcactttgggaggccaaggcaggcggctgggaggtgtaggttgtagcgagccgagatcacgccactgcactccagcctgggcaccattgagcactgagtgaacgagactccgtctgcaatcccggcacctcgggaggccgaggttggcggatcactcgcggttaggggctggagaccggccaggccaacacagcgaaaccccgtctccaccaaaaccagtcaggcgtggcggcacgtgcctgcaatcgcaggcattgggcagactgaggcaggagaatcaggcagagaggttgcagtgagccgagatggcagcagtacagtccagcttcggctccgcatgagagggagactgtggggagagggagagggagagggagagggagagggagagggagagggacttctcttgaatgtcttcacaatgtttaaacaacactgcctgttagggcattattggtactagcaaaacagacaaaattttttcagaaagctagaggcaacccgaatgcccatcaatagataaataattacacatatcatagcaaataccgtaaattggcacatataatgacacagtgaagacatgctgttcatgaggaaacacgaacagcatatccaggataaatcctataaatgggtacttttctggaagaaaacataagaaactgttacagattaccttgggactctgagaaagtggagatttattcttcaattttacacctgtctacattgatttggaattttctaacaatgtgctattacttttgaacggtgaaaaagaattatctggtggaaagattgtggaccacgttttccttatgcttaaaaaatgtttttaacaagaatattttgttgttttaaatgggtagtagattataaagctcaacaaatcatggcaatataaccaaatatagatggaggaatcttgctcccaccatcatccccccatgcactctactctttccagccctctcccagcggacctcccattagatcctgtgagtaagcagttttattgttcacgtatcctttcagcgtttctttatgcagagtcaagcaaatacaaatgtcttttctggccccccccccccctttttctttctttcttttttttttttttttgtgagacagagtctcgctctgtcacacaggctggagtgcagtggtgcgatctccgctcacttcgatctcggctcactgcgatctccacctcccgggctcaagcgatcctcccacctcagccacccaagtagctgggtctacagacgcgcaccaccatgcctggctaatttttgtatttttagtagagacgggttttcgccgtgttccccaggttggtcgtgaactcccgagctcaaacaattcgcccacctcaggcttccaaagtgctgagattacaggcgtgaggcaacgcggctggcttttttttttttttaacttttatgttaagttcacaggtaaagtgcaggtttgttacataggtaaacttgtatcatgggggtttattgtatagattatttcacaacccggatactaagcctggtacccattagttatttttcctgatcctctccctcctcccactctcctccctctaatggaccccggtgtgtgttgttcccctttatgtgtccacgtgttctcatatttatttcccacttgtaagtaagaacatgtggtatttggtttcctgttcctgtgttaatttgctaaagataatggcctccacctccatccatgttgctgaaaaggatatgatctcgttctttttttatggctgcatagtattccatggtgtatatgtaccacattttctttatctgatctacctctgatgggcatttaggttgattctatgtcttcgctattgtgaatagtgctgcaatgaacatatgcgtgcatgtgtctttatgatagaacaatttattttcctttggttttatacccagtaatgggattgctgggttgaatgatagttctgtttttaggtctttgaggaatggccacactgttttccacaatggttgaactaattcacacacccaccagtagtgtataagcgttctgcctgcctttcttttttttttattttttattttttttgagaccgagtctagctctgccgcccaggatggagtgcagtggtgcaatctcggctcactgcaacctccatctcccaggttcacgcgattctcctgcctcagcctcctgagtagctgggattacaggcacacaccaccatgcccagctaatttttgtatttttagtagagacagggtttcaccatgttggtcaggctgatcttgaactcctgacctcgtgatccaccctccttggccttgcaaagtgctgggattacaagcatgagccactgcgcccggccatgcccaccctttcttatacaaacgtagcatactctgttactgctccacaccttacttttcccacctaacaatatgtcctggatatctttctaccttactaaagaaatgtcttttcccatctccctcttttcttctttttctttttttcttttttttttttttttgagacggagtttcactcttgttgcccaggctggagtgcaatggcgcgatctcagctcactgcagcctctgcctcccgggttcaagcgattctcctgcctcagcctcccaagtagctgggattacaggtccaccatgcccagctaattttgtatttttagtagagatggggtttctccatattggtcaggcaggtctcgaactcccgacctcaggtgatccacccgccctggcctcccaaagttctgggattacaggcatgagccactgcgctcagcctcccatctccctcttttctaatgtaagtatttcatgctataaatttccctcctagtaatgctttagccatggcccgcagattttggtatgttgtgttttcatttaattcagttcaatgtagtttttatttcctttgaatcttaacccatggattatttagaagcatgttgtttagttatcaagtgtttggggattttcttgctagctttctgttagtggtttccattgcagtcagaggacctacttgtatgatttcaattcttttaaatttgttaaggtttgtcttatgtcccaggatgtggtgtatctgatatatattttgtaggcatttgaaaagaatgtatgttcgctgttgttgggtgccgtgttctgttggtgttggttagctccagttggtcgacggtgctagagtttttctatattcttctatttctctctatttattctatcaattgttgatgaaggggtgttggagtctccagctagaattgtagatttgtctatttctctttccagtttgatcagtttttgcttcacatgctttgcagctctgctgttttatgtaaacacatttagaattgctgtgtcttcttggtagattgaattgctatgtcttctttgtagataagattggcccttttattagtaagtaatatctctctctgtccctggaattttttttctcaagactgctttatatgatttttttttttttttgagactcagtttcgctcttgttgcccaggctggagtgcaatggcgggatcttggctcactgcaacctctacctcctgggttcaagtgattctcctgcctctgcctcccgagtagctgggattacaggcacccgccaccatgcctagctaattttttgtatttttagtagagtcggcgtttcgctatgttggccaggctggtctcaaactcctgacctcaggtgatccacccgccttggcctcccaaagtgctgggattacaggcgtgagccaccgcacctggccttactttatatgatattaatataaccactcctcttttctttttcccccataaactcaaccaagtggtaccttctttcttttgatgagtgtgtttgcatgatatattttttccattcgcttactttcaatctgcctatatttttatatttgaaatgaatttcttgttgacaatatagaattggtcatgttttttattccactctaccaatctctgtcttttaattgatgcatttaaaccatttatgtctaatggtttacctgccattttattttctgttttctgtttgtttcctctgttttatctttctgttttcttttccttgttagttactagaacacttttttatggtgaaaagttaagatatacatatatttagaattaacctgctggactcagtttagatgatcccaattttgttggcaacatccaaagcattgtaatcaggagccagtcaaatatatgccttcttctctccatcagacctaatcagggtgttgaccttgggcatatcaatgtcatagagcttcttcacagcccgtgtgatctggtgcttgttggctttaacatccacaatgaacacagtatgttgttgttttatatcttcttcatggcagactccgtggtcagcagaaacttgaggatagcatagtgggcaagcttgtttctccttggggcactcttctgaggatatttgggctgcctccagagtctcagtgtcttgggccgccggaaggtgggtgacgtgcggatctttttttttgtggctgtggacacctttcaacactgccttcttggccttcaaagccttcactttggctttggtttaggaggggcaggagctttcttcttcacttttggcaccttcttgtgaaaaggggttactggaacatttatttggaaatccattttggtttatctgtagtgttttggagtatatctctttatgcttttttagtaattgctttaggtattacaatatatagacttaaattatcacagtctactggttgagcatagtaccacgacaaatgaagtatagaaaccttaccttcctttacatgtctttacctttctgtttacggaacttcctctatctgttcttttaggggagatctgctggtgacaaattctctcaattttcctttatctgaaaatcttgttttttcattcattcctgaaaaaaaatttttcactggacgtggaattctgagttgacagtacttttctttctgcacttaaaaaatgttgtgtcaggccaggcgcggtggctcatgcctgtaatcccagcactttgggaggccaaggcgggcagatcacaaggtcaggagttcaagaccagcctggccaacatggtgaaaccccatctctactaaaaagtacaaaaattagctgggcctggtggcaggcgcctataatcccagctactcaggagactgaggcaggaaaattgcttgaaaccggaaggcggaggttgcagtgagcccagatggtgccactgcactccagcctgggcaacgagagccaaaatccatctcaaaaaaaaaaggaaaaaaaaatgtgttgtgccgcttccttctggctttcatgatatctggggagaaatttgttgtcattcgaactgtttttctcctataggcaaggtgtcctttctgtcttattgttttcaagattttttttcttaacaacaacaagacaaaaagaaatcttaaggtaacagaaaggttgaaaaaaacaagatttttttctgtctttagattcagaagttttactatgatgtgccttggcacaagtttcactgggtttattctgtttggcgttctctcagtttcttgaatctgtaggtttatgtcttttgccaaatttgggcaactaaagggaggactgtagttgtaattgtgtcttcagcatacatttcattcctagtgacccatccagacaccatcctttcctcctcccatcttaacttttttttttttttagacagattttcattctgtcgcccaggctggagtacagtggcatgatctcagctcactgcaacctccatctcctagtttcaagtgattctcctgcctcagcctcccgagttgctgagattacaggcatgcgctaccatgcccagctaattgtttgtattttttttttgaagagacagggtttcaccacgttggccaggctggtcttgaactcttgacctcaaatgatcctcctgccttggcctcccaaagtgctgagattacaggcatgagccaccatacctggcctcattataacttctaatattataaactaattttgtctggatttgatgcagtaagacagcatatgctattttgtgtctggcatctttcattgtgcaattcatttgtattattgcatatacttgtatgtcgttcactcattctcatgactgcctactattctatatgagttgatgaataccaacatgtatttgtccattctactactgggacatttaaactgtttccagtttgggaatatgttctctctgcaccagtcatgaatagtgttgctctaaacattctagtatatatcttttggtgcacagatgaatgcatttctgttggatgcatatccaggaagagaattcctgggtcataggataggcatctgttcagctttagtagataacaccagttttccaaagtgattgtgcctatttgtatgctcatcagcagtgtacaagggtttccacgttccacatcccctgtaccagactcttttcatattcgttcttcctagttcttgaaacaactctgtcgggtagacattattattcgcattttaggaatgtggaaatcaaggctcaatgaatgtagatgatttgctgaaagtcacatgcctagtgtggcaatgtttcttggctgggcagaacaggagctctgttgaaaccctccctgatagaatgaggcaacactcttctcaaagaaaggttctctgagggctctaggtaaaataagactgctaaagacatatgtccactctggtttgcaagctacccagaagctctgtggtagagaacttgagcttggcctattattggcatttaaacatgtgcagctggtggagtggaatttcctgaaggtcaaactggggtaaaggagggaaaaaagagggagaaagaggaggaagaggagaaggagaagaagaaaaagtagaagaaggaggaggaggaggaggaagaaggaagaagaagggaggagaaagaggaagaggaggaaaaggaggagtgaggggacatacgggaggaaggaggatgggcccttgaggcaaagtcactgtgtataaaagacatctggtagagaaggctttactgggcagcagagctgggatattagaggctgtaaatccaggtgagaagtgaggctgcacttgactgtcactgtaacccagctctctgcctcacacccacaccctgggactctgggaaggagagaactatgatataaggaaatgatgagtattactagggaccagcaggttagctgcgggaatgttttttcaagtagccgatctgaagaagtagatggaaagtttctgttggctggcgtaaaagggagacagaagccacagaagtctggatgattactatatcaaaatcaaaagttaatcaaaatcacttcctcttcctagataacactaggattttcaaatactttaactccattttcccctgcccctaatttatatattatagttggcaatattttaattatttaccccccacaaaacataatttttattttatacataaatatatatataaataaaatacgtgtgtgtctgtgtgtgtgtatttgcagttagtattcatgtagatttatctacatttttaccacttttcttcttctttaattcttcttaggttttcttagttcttacatccaacatgtgagatgatcactttcttctttcagaatttttcttcactgaggtgataaactttgttttgatcataaaacaaattttttatcttatttgtaaatttcctcttggtattaaaattacaagttggttgttactttcagcacactgaaaatattctaccattttctggtttccattttctattgagaagtcagctattagactgccatttctttggaagtaacctgtctttttttccagctgctttcaaatttttttcgctctttggtttctgcatttttcactgcaatgtttctaggtatggtttcttctcatttatccccctatggattcataggcttctcagagatagacattgttatttttcatcagttccagaaaacctcagctaatctcttcaaatagtgctctgccccatcctctctctactctccttctgggattctatttaaatatattttagactttcctcctctgacttctatgtctcttattctctttcattgtcttttatattttttaatctccctgtattacattccaggtaaattcttctgacctaatgttcagttttcttttcagctttatctagtctgtcattaaattcatctattaagtttttaccttcatttctgtatttttcatttctagaagttctattcactttttttcaaatctgccatgctgctttttatagtttcctacttgctccctactttttcaaacttatcttatttatttaaacatagtaaacatattgttacatataacgtgtctgataatgccgatttctgaggttttcatgggtctttcctgctagctgtctttctgctggttcttgctcatggttcctagtttccttgtgtacctgttatcttcaagaatgtgttgggtactattggaataatttgtgaccttggaagatgataactttcttcagagactttcatatttgcttctgccaggcacctgagatcattactggttcttaaaccaagttcaaggtttgatactctctggacattcagatgaggaacactttgactataaatccatgtaagtttgcttctggattacactcactctgaggttgtggttttttgagatcccagctttctatatacggggtctcttatcatactctcaacttgtgtgggtcctgggcccttatcaccccatgtggttctcgctagaactggcaaatgcccttgaacaaaaatcacttcttttcttatgagcttcactgtgtgcccattttcctttcagttaggcctagtaagtctttgctatcttgtcttttcagtgcttttaagaagatgctctttatattttatctagcattttaagctgtttttagtggaagatattttctgaataacatagcctgccaaaactctcaaatattatttaagcaaagtgtattattccctttcctcccagaagacctcacttttgaaactcaaagccagaagatgctcctgtttgcattgtgtcatttcttccctaaggcaataaactaagtatcttccaagattaagtgagaaaggggctaccggataacagaaagtgccctccccaatacggatactatcatgaaagattttctgccacatgcctaacatgctttccaggacatagttagggctcagtaaatagtttctgttgtcatgattactatttctatatctaaactcgccttgtttaggaagccttcctttaataaccttgtcccattctgatctttcatttactctattttctccaagtactttatttatttatttattatttattgagacagggtctcactctgtcacccaggctggagtgcagtggtgccatctcggctccctgcaccctctactgcctgggctcaagcgattctccttcctcagcctcccaagtagctgggacttacaggcacatgccaccatacctggctaattatgtttattttttgtaaagacaacgtcttactatgttgcccaggctagtcttgaactcctgggctcaagctatcctcctgccttggcctcccaaagtgctgggattacaggcgtgagccaccacggccagcctctccaagtatttttttaccctcattgatagcttattgattcactctagctgtttttcaaatatgcactatttctcaggaaaaagcaatgactgaacatcccccagaagtctttgagggtgtctaagtcctctgtttctttatttctcttatttatttatttatttgtttttgagatggagtttcactcttgttgcccagactggagtgcagtggcacgatctcggctcactgcaacctctgcctcccaggttcaagtgattctcccgactcagcctcccgagtagctgggactacaggcatgcatcaccatgcccagctaattttttgtatttttagtagagatgggggtttcaccatgttggccagactgatttcaaactcctgacctcaggtgatctgctcgcctcagcctcccaaagtgctgggattataggcatgagccgctacgtccggccaagtcttctgtttctttgttaactctgatatcaattagaattctttcagctgcaagtgacagaagactcaattcaaactgcattaagcaaaaaaaaaaaaaccattctattggttcatgtaactcaaaaaccaagtggtggatcttcaggtacagtttgatccaaggacgcaatgaatgtcaggagaacctggtctttttagtcatagtggctattttgccttttgttttggcttcaccttcaggcagctccactctgcatagtagcatccagaatctccacacttacatccttacagttccaaaggcagtgggaaagagtttctaactccagcaaaagtcccaggattcacccctattggactaattctgtcaatgcccacttcactgtggccagaggcatttggagtgctaattggctacaactgaaccagaggaatacttcgtggaacccacagttgagcccttctgaaaatcatgtgctgcaggtgaattgagggaatgtggtttcctaaaggaaaacccagaagtttgccaaaagatggggtaaatggatactgggtatagaaaagcaacaactgtccactacaattcccaattgctcacagaaaaagactcagtaatggggcctgtggcgcagactaaattgcattaacaaggtgctggttctttccaggtccttcagacatttgcacagatgctgctccgtctagccacacacaaacaggacagagcagttgtgtcctgtgatggatatgaaggctgctctcccagcctcccaactgctctggcctaggggaccctatttgtgatttattcagtaatgggtctcatccaggctactctcttggcctcctcaagctgtttttcctcctcctgatataaaaataacacttaaacaggttccccaaccccatgtccgagacaaatcagatctccagagatagcagccaaattcaccctagggtcagggtgcagggtccttattatatacccagtctgcgagtgagccaagaagtagggtttccagaaatatgatgccatggccagaggctaaaatcaatcacccgaggagtatttattacccaactgaagccagtacctcctgctgatttgccctgccagacgatgagtggaggcaggcttgtggtcagagcagggtggagatctgagaggcgaaaggcaggtgtagggtgagagaaggggcaaggcagagctcttcttgaaaactgtgaagttgctatgcttttagtgttgagcagtgctggtgtgtacccccagctggagtcctcctccaccccaaccttgacagcagcaggagatggctactgaagggtcagagccctcccgtgcctgcctgcctgcctgcctgccttcaaagcctcatggttggaggtatagagggggcaggtctgtgtcttccataaagcttagtggttataaatgtggtgcctgagtctgggtttgattcttggctccatcccactagctttgagattttgatcaagttacttaatctatttatgccttggttttgtcatctgtaaaattgtaatagtcaaagtatttccctcacagggttgcaagggattacacaggacatggcatataatgtgttttgcacagtgtgtagtagacactaagcactgcacgaacatgcagggtcattatctacatatgtaattcctaatcctacctcaatcctgcaacgtagatgtattagttagagagaggcttggctgctataacaaaggttcaatttgtcgatcgtttaaacaagacagaaatttatttatctctaacataacaatccagaggtaggcagaccaggacaggggtgggggatgttctatcattttcaaactgcattttccatcgttgggttcagtttgctgctccggctcccatcatcgtattgtaattctagccagtaagaaagggttccctttcctcctaggggtacccttctatcatataatatatatgtatataacattgctgctcaaatttcatggtcaaacttagtcatatggccatactatctgcaaaagaagctgtgaaatataatcttgcattcagttaaaattgtagtggtttactaacagcagaagaagggtagaagagatattgctctactagagctcacccatctggccatcccacacaaaaaacacagttactgcccttcaaaaggaggcaagtcagagttccatctgagtacggcacccagctgagagtccaggctatctgaggggtgggcagttccttcttccaggtagatggacaccctatggtctggtgacctaaaaactaacaggcaggtgatttgcacctcctactccccacagacaacagtggaggaagaagagggtaagctcaattaaaacactgactcatgctgagcacactgctacgcgcctatagtcccagctactgagataggaggatcattgagcccaggagtttgagactagcgtggacaacatagtgagacactgtctcaaaaaaaaaaatccacttgcaactgaaagaatggcaaacacccagcagctgctggatcttaattatcaaatctggttgggcaggaatggctaagccctttcctggcagtggagtaagttcttggtcagcacatttgaccacccctggttctgctctctgggaagatctcccctttccactctcctatgtagcctctggcttttcctgctgggaagctgttgtttatccaatatcctttgtcccacactggcaaacttgtgctttctgcagtccttccagccttcacagccctcttcctgctggtgcggatttggagggctgggaataaggcaagggaaggggtgtcaaaccaggctttgctgatcaggcttatggtttctttgccaatacaacaccctcacaatcttagaagccttctgagtggctccatgtttgagtaaccacagccaatggcctcatgtagaccttatttttatgcttgaacactctggacttggtgttcttcctactctcctttctctcaaactggtggtggctatcttgagtccatttgaaaaaacatgcttggtgtgaaggaaattctcttacactgatctctgtcctctgagccagttcccactgtccagtgagcagatgtccttgagaaaggctggggaccacaggaacagccctggccaaggctgtcctcccaactccattctcctccaccctccccattggcagagtaggaacattgagggttgatggaaaacacatgtaataaactcagacagaaattcaccagtctgttgttgagtgtccactccagggccagggctgggatggaagaggctggaaggaggagagaaagggaggggcagagagaggaaagccggagcagctgggtcagcattcccaatccggacattgactcaggctccgggaatgccaaggaagagggtggcagctactggagccaggctgggatcctgttggcaccagggatgtaggcacagcactcacagagggttgggggctgggacacctgcacacccaggcatatctcccaaccacaggtggaaacagatgtctcttcccctgcggggagctctgagtgctccccacgccttcgaggtgattctcacagctcttcacctgcctgaaacacccactccatctccttcacctgagagagggacacactccagggactcaaatctggacatctggacatacactagctgctctcctggactacccacaaaccccttgcctcatctccagcctctgagctggctgcaacttgccctttctaactccagcaccaccctctactgaaattagaacaaggaagaaagttatgctgatctgcctccatgttcccttctttggtgcctgataaagatgagggaggctcttggaataggggctcaggactctgagagcccaactctgctcaatgaccatgttcccacatgctccaagccacatcccctcaaaaagggtccctctagcttgtcctcagtgacccaggaggcagctgaggaccaagtacccagattatccggtgcgccccttccctcccagcaacccccagccttcagggctgtagcagctgagcaaatgggggcccctccctctcattgcctgacacccaatcagagagaaaccgatcctggcagggcagggtgcccggggccgggcccagaatagtgcagcccagccacagtgtcgcacacttgctctcagttggtctggggctggccacatggagcccgggctggagcacgcactgcgcagggtatgggggtcccaggggagccggagccggggcagctgaggccagaagattgagcgcacgggctgtgaatgtgtgtgtgggcgtgtgtgtcttctggtgtgtgtttggtctggattttctcgtgaatatgggcatgtgcatgtttgggcatatgtattgtgagtgtgtgtggttctgtgtgcctgggagtgtttggatgtgtgtgtttctgtgtgtgtttgtgtatggctgcatgtctgtgtatggcgtgtgtctgagcgtgtgtattggtgtgcatgggtgtgtaggcgtgtgttcagggagaaggggtttgggaatgtaaggcactttccccactccttcagaaactcttctccccacagaccccttcctggagcagccttgggggttctgagcatcaaggtagggagaatgccccctccctggggcctaacctcttcccccacctccttgtcccccacttttctgggaccccaattccctcccagcctggcttttatcttcctccttttggtctttcttcctcattgttcttcctcctttccccaggtgtgtttccctcacctccaatttcctcttttcagaagtgactttcccacttacttgctgtgtgatttgcagcaaattgcttaacctctctgaatttctggtccctcactagcaaaatagggatgataataatgcctgctttataaggctgctgtaagttttaaatgagaaatatgttggagaaaagcccattggaagcttgcaaatcctaaagattatgaataacatttgaattgattttgatgcattactccctattaaccaaacaggccccattccccttccagagatgagcttcctagagcaagaaaacagcagctcatggccatcaccagctgtgaccagcagctcagaaagaatccgtgggaaacggagggccaaagccttgagatggacaaggcagaagtcggtggaggaaggggagccaccaggtcagggggaaggtgaggccaaggccagttctggggaggtgggagccaggggagtgggaaatcccagaggagcctgggtctggtctctacctcaggtccctccataacacagagttggacccaaccttcatcttgtggcctcagtctccctacatagtagagaacaaggcactgcagtgccagaggccagcatggccaactcagaaagatgggacagagccactacctggggcgactctcaggtcagcccctcacctgcaaatagggccacagcatccaggcttcccactgctgctgtgagatgaatggcgacagcagatgagaacgtgctttggaagatggagttactgtcctcttcccctcctcccccaaacaggtccccggtccaggccagctgctgagtccaccgggctggaggccacattccccaagaccacacccttggctcaagctgatcctgccggggtgggcactccaccaacagggtgggactgcctcccctctgactgtacagcctcagctgcaggctccagcacagatgatgtggagctggccacggagttcccagccacagaggcctgggagtgtgagctagaaggcctgctggaagagaggcctgccctgtgcctgtccccgcaggccccatttcccaagctgggctgggatgacgaactgcggaaacccggcgcccagatctacatgcgcttcatgcaggagcacacctgctacgatgccatggcaactagctccaagctagtcatcttcgacaccatgctggaggtgaggccacggctctgcccaacctgtactcactctccatccacacggtgctgcagccgccactcccaccctgcaggatgccctgctgagccaggtgcccctgcaagccccctgaaaggactccttcttagcactatggaggccagttgggggagggacagcttctactctctgttagcacacgtaattgtcatcacagctctgccacttagtagctgtaggaccttgagttacattacctttcttgtctctaaaatggggatcgaaatgcctgctgcaccctacatagttcagaggattcttggactacccagtataaagcgtgaacaaagtatgtgtaagacattgcagaggccgggcacggtggctcatgtctgtaatcccagcactttaggaggtcgaggtgggcaaatcacaaggtcaggagttcgagaccggcctgaccaacatggtgaaaccccatctctactaaaaaaaaaaatacaaaaaattagccggccgtggtggtgggcgcctgtaatcccagctactttggaggctgaggtaggcgaatcacttgaacccaggaggcagaggttgcagtgagccgagatcgtgccactgcactccagcccgggagacagtgcgaaattccatgtcaaaaaaaaaaaaaggcatcacagagtgacttagaaaaaagagtaggggactctcttggtaacagaaaaaataatgagctctggcacctgctcttccattaggcttagctataaatgtctataatcttattcttatcattgttatgcagtgcagtgtttcccaaaacacggtgtatgtattgtccatagcatgcattttgattctaatggttcgtgaaaaaatatgtctatttcagtagctttatatttgttttaggctgtgttaaaaaaatataaccagcatgtgcagatagtactgcttaggacggggcacaaaatgagtcaatataaaatttatctacaaaaaaagtatgtaaatatgcaagtgttatgtaaataactatgcctccggtacttagtctatcaaaaactagactgtgtctgtacacacataccctccaaatggaaacatgaacttagaatttctcactccctggtgtgccaggtgaggggctcatggtctcaggccacagccttatctatgtacatgtccccagcaacgatcatgtctagcgtgtactgtgacatgaggaagacaggccagctggtgtagaggagggtccaggagaaggccccatggaagaaccctgggtgggacagggaggggacagcaggcagatgggaggtgcgcactgaggggcagagaggggggtgagggtctctcgggacccacccttgactgttctccctggcccctcagatcaagaaggccttctttgctctggtggccaacggtgtgcgggcagcccctctatgggacagcaagaagcagagctttgtgggtgaggagaggctggggaggtgaagggagatggaggaggtgagggggagatcttgtacggttgttctggggctgatctctgatataccacaagcttggcttcaggccaagcccagccaggggccagggtggaggaaagtccatccggagtctgcatggccagctgggagaccctggggctcaatttccccatctgtggagccgctatgaccagctgacacctttcacctccgctactgcatggccctgtgccataggtgctagggagcaaatggggggaggcaggagagaaagagccccacttctcaggcctggggggctgccccactgtcctgttcccacagtccccactgtgtctcagcacaaggacactggcagggtggggaggggatctgaccctcaacctgccttccacccaaaggccccgggctgacctcctccccgcccctcccctgcagggatgctgaccatcactgacttcatcctggtgctgcatcgctactacaggtcccccctggtgaggagtgggctgggaatcttatgggcacccagaggggcgggggcggaggggagtcctcctggagcctggtgccctagaagcccacgtctttctgacttctggagtcctgtcgatgtctctaggtccagatctatgagattgaacaacataagattgagacctggaggggtgagtggggagaggaacccggaaagggctgttggtgatggtgggccagggcttaaggtggaggatgggcagtggggatgtcctggagtgaacaggggagggacaataggagcctcgggtgcctgacggaagggaagctgcctgggactgcaaggtgaggcaggtgaccggctcccctggcctgactctggctctttctgcagagatctacctgcaaggctgcttcaagcctctggtctccatctctcctaatgataggtgggtgtctctgctcattcacctgagcctcctcctcccacagtccccttccccagtcccactcagctctgaactcacctcttcatcctaggcggcacacagacaagggagccttggtgccctgccctcctttttaggggcctgggatggaggttgtctctccctaggctgccccgaggctcactgctcccatctctgcagcctgtttgaagctgtctacaccctcatcaagaaccggatccatcgcctgcctgttcttgacccggtgtcaggcaacgtactccacatcctcacacacaaacgcctgctcaagttcctgcacatctttgtaagcctgggcccaggtgggaggaagggggagacctgggcaggtgatcagagggcctgaggagtcttcagccctagcagtcgtggggaagagctgggagccctcttgaagctgctggatccctgatctccacctggtccccatcctaaccagggttccctgctgccccggccctccttcctctaccgcactatccaagatttgggcatcggcacattccgagacttggctgtggtgctggagacagcacccatcctgactgcactggacatctttgtggaccggcgtgtgtctgcactgcctgtggtcaacgaatgtggtacccacccccaggatgagaggctcgggctgggctggggcctgggagaacctggtggggagaatatgggaagggcagggtttctcatgccatccctgtggggggtacaggatggactggggattagaagtctccgtctactctgagacttgggcaagttgcttagcctctctgtgcctcagtttcctcctttgtgaaatgggattcttcacaacactcatctttctgacttcacaggaggatttaaaaggttatgtgcatgaaaatgggcatagcgagaggaagcagcagattttagtttcagttcatcttaaaatggagccggacacgtggtgcatggctgtaatgccagccctttgagagaggccagagcgagaaggtcgcttgaggccaggagtttgagtttacatacagtgacactccaacctgggcaacagagcaagaccctgtctctaaaataataataacgaaataataaaatttaaaaaattaaatgtgccatgggacctcgggcccccagccagccctcaatttcctacactgaaaaagggctgcagaaatgacaagattatttctttggttgaatgatggctatgaaatatttcaaagctaaaatcaccataattaagatagatatcaagaagtgttttcaatttatttttgattataaaaaacttcaaatgtatagaaatggtagaaagaaattgcataatgaaccacctatatccattgcctagattaactactgttactatattgccataaccagttcatctttttctctgaagtaattcaaagtaaattacagacatcatgtcatttcacccctaaacactttagtctgcacttcttaaaacaagtgtatttttggccaggggcggtggctcatgcctgtaatcccagcactttgggaggccaaggtgggcggatcacgaggtcaagagatcgagactatcctggccaatatgatgaaaccccatctctactaaaactacaaaaaaaaattagctgggcatggtggcgcacacctctagtcccagctacttaggaggctgaggtaggagaatcggttgaacctgggaggcagaggttgcagtgagccaagatcgcgccactgtactccagcctggcaacagagcgagactccaaaaaaaaaaaaaaaaagtgcattttataccaccaaacaaaaccatgaataattcatattattgtctgacatccaatccttattctcacttctccatttcctcaagaatgttgggtatttttgttgttgttttgggatggagtctccctctgtcactcaggctaaagtgcagtggtgccatctcggctcactgcaacctctgcctcccgagttcaagtgattcttgtgcctcagcctcctgagtagctgggattacaggcaaatgacaccacgcccagctaatttttggggggtatttttagtagagatgaggtttcaccatataggccaggccagtctccaactcctgacctcaagtgatccacctgtcttggcctcccaaagtgttgggattacaggcatgagccaccgcgaccggccaagaattttttttttttttttttttgagacagagtcttgctctgtcgcctgggctagaatgcaatggtgcaatcttggctcactgcaacctccacctcccagattcaagcaattctcctgcctcagccgcccaaatagctgggtttacaggtgcgtgccatcacacctggctaattttttgtatctttagtagagatggggtttcactatgttggccaggctggttttgaactcctgaccttgtgatccacccacctcggcctcccaagggaatgtatttttcatagttgatgtgttcaaaacaggatccaattgaagtccatggtttacattcgtgtcttttgttccttaaatctctttaaatctataacagctccattccccctaaattgttatgacgtgggtgagttgaaaagcctgggtcagttgtcctataggataccccacatccagatttgtctgtttgctttctagagctggcctttactttgttctctattcctgggtttcctgtaaactgaaattatatctaaaggcttgaagagatttgggcaataaattcgaggctagactattttgtaggtggtgccatgttactcacactgcatttcctcaggatgctcaatgtcaggctgtctcactctgggatgctaagtttgatccttgggttctggtagtggcaccctgatgcaaatagccctaggccctctacacagcaccccggttctgaccggagcctcttccctgtctttctccccccaccccccacaaccaccctctgcaggtcaggtcgtgggcctctattcccgctttgatgtgattgtaagtgtcgctggaaaggtgggatgctgcagggaggctaagggtgtggggatgggtggggggcctctgtggaccagggggaccttgacaagtatgcaggggttgacatctgtagggtaggagcccaggcaagggggtgactaggagccatacttctctctctgccccagcacctggctgcccagcaaacctacaaccacctggacatgagtgtgggagaagccctgaggcagaggacactatgtctggagggagtcctttcctgccagccccacgagagcttgggggaagtgatcgacaggattgctcgggagcaggtaccgtgtgccctccattcatgcccccaacacatatagcccagtccttctcatgcacggctccagccatccctgaacatcgggcacctggcctatccttccatttcatgaccaactcctggtgcccacactggcctgcacctggtcctgtccatggggcccttatgccaggggtcactgccaactgatcaccttaggccggtcacaccatccctaactggtttctaggagacgctctctccctcagtcatgttgggttgtttcccctgattcttggcaccaacctcagtagctgctgtagccccatggctctgccccctcactgaacattgcggacccacaggtacacaggctggtgctagtggacgagacccagcatctcttgggcgtggtctccctctccgacatccttcaggcactggtgctcagccctgctggcatcgatgccctcggggcctgagaagatctgagtcctcaatcccaagccacctgcacacctggaagccaatgaagggaactggagaactcagccttcatcttcccccacccccatttgctggttcagctatgattcaggtaggctctgccctgggccatgacaccagcctcttagtcttcctgatttcctggatctcagattgggacaccctgaggatgggagtggccaagctcaaagctgagcagccctgtgaaaactgcaagtgtaaggactcacaggtccccactttgtcccattttcagctgaggtcatggagactgtcctagaagggagggacaaagccccggctaaaagctaaggctgcgatgtgacttcagacactctgggaagatttacttgttccctcacaattcaatttccacctgctggaaatccccttagaaggaagcaagctatatcaacatttgttggatggaatttccatccccagtaagaactaaagcttaaaggaaggagcttctacctacgtgctcatctcaggaaatgtgacatgatctcagactctttcctggtcacacgtttgctaggggcttgcagaggatgggcacagattccatctactcaccatggctggcaagggtcccctgttggaaaggccgctgtccctcctatgtggtcaccatcttcccttttcttttccccacaggcttcttcagccctcccaaattgcccttgccctacctgtgctcccagaagccctcgggcatgcccagtgcaccatgggatgatgaaattaaggagaacagctgagtcaagcttggaggtccctgaaccagaggcactaggattaccccagggccatctgtgctccatgcccgcccatccccttgccgcctgactgggtcggatggccccagtgggtttagtcagggcttctggattcctcggtttctgggctacctatggcttcagccttcagctcctgggagtcccagctgttgttcccagcaacgtcgccactgccctcctactctccaggctttgtcatttcaaggctgctgaaatgctgcatttcaggggccaccatggagcagccgttatttatagaactgcctgttggaggtggggagtcctccctccattcttgtccagaaaactccttagctctcgcagtgagccatgttcttagtctccagggatggatggccttgtatatggacccctgagaatgagcaattgagaaaacaaaacaaaaggaacaatccatgaacttagattttattggtttcactcaaaatgctgcagtcatttgacctgaacttgtggcaagagacttgtgctttctaaattcaaagactagaaggaaaatggataaaaatcacaagtgccgtttctcttgcaatgtagcgctattctactgaaatttctttcttctcttttctttacaaaatcataaagaaaaaattaattcattacttatatagtaggtacaactcagcctacaaactctaatctgcaagaagcataactttatttttctaacacagaatgtaatttctattaggaaccccgtttcagcaggtggtagaaattaatctcagtcaattcaaagtctccccctgaccttttcctggggttaagctcggtcgggtgggggtgtggctttaagtcatgtaagactctgttcctcggctatcatcatccgtccatgctcgcacctgcctggacatcccctccccatctggtcatcagttggtcatgcaaggtctgccaggggctccttcacttccacaaagcctatttggggacctgtggcttggagcatgtggaagattctagctcctggccctgcacaccctctggcttcaggaagctcagtaattctgttccaggccaactgctaagatagacagcccagtcccttgttccgctctgtggcttgctgttttcccaggctctacccaggaagcgtgtgggttcttctacccagtgcttgcctacacttatcacctccaaacctatatggagtatttgtcatactttccccctctttagccgcaacctccttctcagggtccttttctcaagaattccagaaatgtcaacattcgccttgctccccaagccctgccaacctatggaatcttgaccagtgggggagagaggaggcttttattctttaacccatcatatattcttccaaatgcactgtaagtgtcctgagttcttgccacccaccttcagaatcaagcttttaaaattccaaagccaaggctcgcttctgctatgtcaccttccctgtggcaacgggcaccacatgtctggctgtccagagaagggggagattactgtctctaaaacatgaccccttccccttacctcacagagcgagaaggcagaggctttgtactgaattggttggaccataaatgaagatgttccctcactagacattttgccaaaacctgagggtcagtgctggctctgctgtggtcccgcccagggaaggtccagggtcttgggctcaagggagaggattggagttgggtgatggttgggtctgggcccagagaggatctctccagtgacagtgttcctggggagcctagggctctgggatcttgtaacaggtcagtcctttgaggctgtgcctctgggtggctctgctctgtgggaccttctctgtaagggcacatgccaccctccctgcctcagacccctcctttggtcacagcccggcccttgcagccatgatggctgctagaaaagagtcacggagtttccaccggggctgcggcggctgcttcttccactccagctctgaatcgaagacgtgccctgtgtgcagcccgctgtgccgcccactctgttcctgcatgtgaacttggggcgggactggttttccaggcactgctgcctccgtgcccccagcccctcctcagacctctgcgggcctctctgggtttctgtagcttccttcgctttcccgtctctcttctcacaaccccttcagagctctcc

SEQ ID NO: 2 - protein, 489 aa, Homo sapiens
MEPGLEHALRRTPSWSSLGGSEHQEMSFLEQENSSSWPSPAVTSSSERIRGKRRAKALRWTRQKSVEEGEPPGQGEGPRSRPAAESTGLEATFPKTTPLAQADPAGVGTPPTGWDCLPSDCTASAAGSSTDDVELATEFPATEAWECELEGLLEERPALCLSPQAPFPKLGWDDELRKPGAQIYMRFMQEHTCYDAMATSSKLVIFDTMLEVKKAFFALVANGVRAAPLWDSKKQSFVGMLTITDFILVLHRYYRSPLVQIYEIEQHKIETWRGIYLQGCFKPLVSISPNDRLFEAVYTLIKNRIHRLPVLDPVSGNVLHILTHKRLLKFLHIFGSLLPRPSFLYRTIQDLGIGTFRDLAVVLETAPILTALDIFVDRRVSALPVVNECGQVVGLYSRFDVIDLAAQQTYNHLDMSVGEALRQRTLCLEGVLSCQPHESLGEVIDRIAREQVHRLVLVDETQHLLGVVSLSDILQALVLSPAGIDALGA